(19) United States
(12) Patent Application Publication
(10) Pub. No.: US 2007/0059808 A1
(43) Pub. Date: Mar. 15, 2007

(54) Compositions and methods for inhibiting white spot syndrome virus (wssv) infection

(75) Inventor: Kurt R. Klimpel, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Aqua Bounty Technologies, Inc., Waltham, MA (US)

(21) Appl. No.: 10/570,574

(22) PCT Filed: Sep. 9, 2004
(86) PCT No.: PCT/US04/29438

§ 371 Date: Mar. 1, 2006

Related U.S. Application Data
(60) Provisional application No. 60/501,614, filed on Sep. 9, 2003.

(51) Int. Cl. C12P 21/06; C12P 19/34

(52) U.S. Cl. 435/91.1; 435/69.1

(57) ABSTRACT

The present invention relates to a novel composition useful for inhibiting White Spot Syndrome Virus (WSSV) infection of crustacean animals, particularly those of the genera Penaeus sp. More specifically, the novel composition comprises a polypeptide whose amino acid sequence corresponds to at least a portion of Vp28, a surface protein of WSSV, or an antibody that specifically binds the polypeptide. The polynucleotide sequences encoding the Vp28 polypeptides of the present invention are also disclosed. Further disclosed are methods for using the novel compositions to inhibit WSSV infection in crustacean animals.

### RELATED APPLICATIONS

This application claims priority to U.S. Provisional Patent Application No. 60/501,614, filed Sep. 9, 2003, the contents of which are incorporated herein by reference in the entirety.

### BACKGROUND OF THE INVENTION

Viral diseases are major problems in the shrimp aquaculture industry worldwide that can result in large economic losses. White Spot Syndrome Virus (WSSV) is one of the most significant viral pathogens. Industry losses due to WSSV from 1995-2002 exceed 8 billion US dollars. WSSV-infected shrimp become lethargic, show a reduction in food consumption, loose cuticle, and often exhibit “white spot” under the exoskeleton. The virus infects most crustaceans, but is fatal only for shrimp.

WSSV virions are enveloped nucleocapsids that are bacilliform in shape and about 275×120 nm in size, with a tail-like projection at one end of the particle (Wongsteerasupaya Dis. Aqat. Org. 21:69-77, 1995). The double-stranded circular DNA genome is about 305 kb (see, e.g., van Hulten et al., Virology 286:7-22, 2001; WO 01/09340; WO 02/22664; and WO 03/070258). Based on the sequence and phylogenetic analyses, WSSV is a member of the genus Whispovirus within a new virus family called Nimaviridae, referring to the thread-like polar extension on the virus particle.

The double-stranded WSSV genome is enclosed in a protein coat that is in turn covered by a bilayer lipid membrane. Viral proteins are inserted through the lipid membrane and project from the surface of the mature virus. The viral proteins interact with the receptor molecules on the surface of the cells lining the gut of shrimp, which brings the viral membrane in close proximity with the shrimp cell membrane, thereby resulting in fusion of the two membranes, which allows the viral DNA to enter the shrimp cell.

The WSSV genome has been sequenced (van Hulten et al., supra) and potential viral proteins identified. Four viral proteins have been confirmed to be expressed and located as part of the nucleocapsid or on the surface of the viral outer membrane. Vp28 and Vp19 are on the surface of the virus. Vp35 and Vp26 are part of the nucleocapsid.

Immunological evidence suggests that Vp28 functions on the surface of the virus to mediate viral infection (Van Hulten et al, Virology 285:228-233, 2001). These studies were performed with antibodies to Vp28, which inhibited virus infection of shrimp cells. The prior art, however, did not demonstrate the region of Vp28 that interacts with the receptor.

The present invention provides new Vp28 compositions and methods for inhibiting white spot virus infection.

### BRIEF SUMMARY OF THE INVENTION

The current invention is based on the discovery that Vp28 is the major protein that interacts with WSSV receptor on crustaceans, e.g., shrimp, and marine insects. The invention therefore provides methods of inhibiting WSSV infection by administering agents that block Vp28 interactions with its receptor. The invention also provides compositions, e.g., peptides or antibodies, that block binding of Vp28 to the receptor, thereby preventing or inhibiting WSSV entry into a cell.

### BRIEF DESCRIPTION OF THE DRAWINGS

FIG. 1 illustrates varying degrees of protective effect against WSSV infection when shrimp were fed with polypeptides comprising Vp28 or Vp35 (at concentrations of 25 grams per ton or 5 grams per ton). Controls were also included.

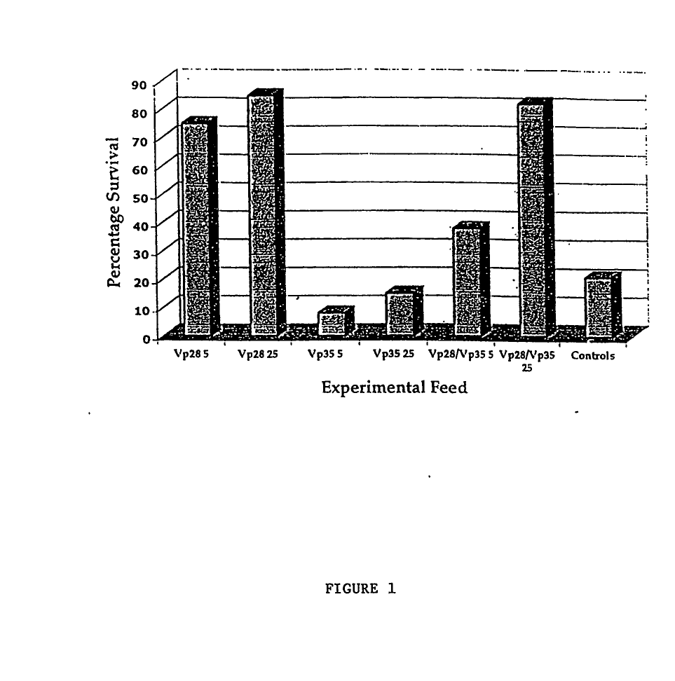

FIG. 2 illustrates the survival of shrimp on different diet after exposure to WSSV.

### DEFINITIONS

A “Vp28 peptide” as used herein refers to a peptide that consists of an amino acid sequence of at least 8 contiguous amino acids of positions 28-204 of SEQ ID NO:2. Preferably, a “Vp28 peptide” consists of an amino acid sequence of at least 44 contiguous amino acids of positions 28-204 of SEQ ID NO:2, i.e., this amino acid sequence may have at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43, and preferably at least 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 11, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, or 177 contiguous amino acids of positions 28-204 of SEQ ID NO:2. A “Vp28 peptide” is encoded by a “Vp28 polynucleotide,” both of which terms as used in this application include naturally occurring and recombinant forms. Also, a “Vp28 peptide” and a “Vp28 polynucleotide” may encompass all variants comprising one or more conservative substitutions, which are described in detail below, given that the variants do not alter the activity of a “polypeptide comprising a Vp28 peptide” to inhibit WSSV infection of Penaeus sp. cells.

A “polypeptide comprising a Vp28 peptide” as used herein refers to a polypeptide that contains a portion of its amino acid sequence derived from a Vp28 amino acid sequence, i.e., a “Vp28 peptide” as defined above, and the remaining portion(s) of its amino acid sequence is heterologous to Vp28, i.e., derived from a source other than the full length Vp28 amino acid sequence.

A “full length” Vp28 protein or nucleic acid refers to a polypeptide or polynucleotide sequence, or a variant thereof, that contains all of the elements normally contained in one or more naturally occurring, wild-type Vp28 polynucleotide or polypeptide sequences. The “full length” may be prior to, or after, various stages of post-translation processing or splicing, including alternative splicing. SEQ ID NO:2 is an exemplary amino acid sequence of a full length Vp28 polypeptide.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine () (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A variety of references disclose such nucleic acid analogs, including, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

Other analogs include peptide nucleic acids (PNA) which are peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C. -95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Sambrook et al., Molecular Cloning, A Laboratory Manual (3rd ed. 2001) and Current Protocols in Molecular Biology (Ausubel et al., eds., 1994).

An “antibody” refers to a glycoprotein of the immunoglobulin family or a polypeptide comprising fragments of an immunoglobulin that is capable of noncovalently, reversibly, and in a specific manner binding a corresponding antigen. The typical antibody structural unit is a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively.

The term antibody, as used herein, includes both monoclonal and polyclonal antibodies, and encompasses antibodies raised in vivo, e.g., produced by an animal upon immunization by an antigen, and antibodies generated in vitro, e g., generated by hybridomas. This term further encompasses single chain antibodies (ScFv).

For preparation of monoclonal or polyclonal antibodies, any technique known in the art can be used (see, e.g., Kohler & Milstein, Nature 256:495-497, 1975; Kozbor et al., Immunology Today 4:72, 1983; Cole et al., Monoclonal Antibodies and Cancer Therapy, pp. 77-96. Alan R. Liss, Inc., 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, 1992).

The term “specifically bind” as used herein to describe the interaction between an antigen, e.g., a Vp28 polypeptide, and an antibody refers to the fact that detection of any antibody bound to a particular antigen is determinative of the presence of the antibody against the antigen, often in a heterogeneous population of other antibodies and proteins. Under designated immunoassay conditions, a detectable signal is designated as one that is at least twice the background signal. Thus, a specific antigen-antibody binding should yield a signal at least two times the background and more typically more than 10 to 100 times the background.

The term “inhibition of White Spot Syndrome Virus (WSSV) infection” as used herein refers to a reduced incidence or severity of WSSV infection in animals of the susceptible species, as shown in reduced number of animals manifesting symptoms of the disease, including death, following exposure to WSSV. Inhibition of WSSV infection is achieved when a peptide decreases infectivity by at least 10%, often by at least 20%, typically by at least 50% or more relative to a control population.

The term “crustacean” as used herein includes any and all crustacean species, which include those commonly referred to as “shrimp,” “crabs,” and “lobsters,” such as Penaeus, Litopenaeus, Marsupenaeus, Fenneropenaeus, and Farfantepenaeus.

### DETAILED DESCRIPTION OF THE INVENTION

I. Introduction

The current invention is based on the discovery that viral protein Vp28 mediates the binding between WSSV and cell surface receptors, a necessary step during WSSV infection. Thus, the present disclosure provides an effective means for inhibiting WSSV infection by administering virus-free Vp28 protein to species susceptible to WSSV infection, such that cell surface receptors will be not available to WSSV. Fragments of Vp28 (as well as their corresponding coding polynucleotide sequences) have been further identified in this invention for their ability to block WSSV binding and thus inhibit WSSV infection. Accordingly, polypeptides comprising at least one such functional fragment of Vp28 can be used to inhibit WSSV infection of shrimp, lobsters, crabs, crawfish, and other crustaceans.

II. Vp28 Polypeptides

Vp28 polypeptides are fragments of Vp28 that have the ability to inhibit WSSV infection. Such fragments comprise at least 8 contiguous amino acid residues from positions 28-204 of SEQ ID NO:2. The polypeptides can be of any length, but are preferably 150 or fewer amino acids in size. Exemplary fragments are set forth in SEQ ID NOs:3-8. Vp28 polypeptides include variants that comprise conservative substitutions that retain WSSV-inhibitory activity, such as Val for Leu, Asp for Glu, Lys for Arg or His, and Gly for Ser or Thr.

WSSV-inhibiting activity can readily be determined using techniques known in the art. For example, a peptide can be evaluated for the ability to inhibit WSSV infection of a population of shrimp or other crustaceans, using methods exemplified in Example 2. Infection is typically assessed by determining survival of the animals following infection. Inhibition of WSSV infection is achieved when a peptide decreases infectivity by at least 10%, often by at least 20%, typically by at least 50% or more relative to a control population.

As appreciated by one in the art, the level of WSSV infection can also be measured using endpoints other than survival. For example, levels of infection can be determined using antibodies to WSSV proteins, including antibodies to the Vp28 polypeptides of the invention, to determine infectivity.

A. Recombinant Production in Prokaryotes and Eukaryotes

Vp28 polypeptides of the present invention can be produced using routine techniques in the field of recombinant genetics, relying on the polynucleotide sequences encoding the polypeptide disclosed herein. Basic texts teaching the general methods of recombinant techniques used in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

Expression Systems

To obtain high level expression of a nucleic acid encoding a Vp28 polypeptide, one typically subclones a polynucleotide encoding the Vp28 polypeptide into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., supra, and Ausubel et al., supra. Bacterial expression systems for expressing the Vp28 polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the Vp28 polypeptide-encoding nucleic acid in host cells. A typical expression cassette thus contains a promoter operably linked to the nucleic acid sequence encoding the Vp28 polypeptide and signals required for efficient polyadenylation of the transcript, ribosome binding sites, and translation termination. The nucleic acid sequence encoding Vp28 may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a Vp28 polypeptide-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

As discussed above, a person skilled in the art will recognize that various conservative substitutions can be made to any Vp28 polypeptide or its coding sequence while still retaining its WSSV-blocking activity. Moreover, modifications of a polynucleotide coding sequence may also be made to accommodate preferred codon usage in a particular expression host without altering the amino acid sequence of a Vp28 polypeptide.

Transfection Methods

Standard transfection methods are used to produce bacterial, mammalian, yeast or insect cell lines that express large quantities of Vp28 polypeptide, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing Vp28 polypeptides.

Purification of Recombinant Polypeptides

After the expression vector is introduced into the cells, the transfected cells are cultured under conditions favoring expression of the Vp28 polypeptide, which is recovered from the culture using standard techniques (see, e.g., Scopes, Protein Purification: Principles and Practice (1982); U.S. Pat. No. 4,673,641; Ausubel et al., supra; and Sambrook et al., supra).

1. Purification of Proteins from Recombinant Bacteria

When Vp28 polypeptides of the present invention are produced recombinantly by transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinlanan Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins, e.g., a recombinant Vp28 polypeptide, from bacteria periplasm. Where the recombinant protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification
(a) Solubility Fractionation

Often as an initial step, and if the protein mixture is complex, an initial salt fractionation can separate many of the unwanted host cell proteins (or proteins derived from the cell culture media) from the recombinant protein of interest, e.g., a recombinant Vp28 polypeptide. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.
(b) Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a Vp28 polypeptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.
(c) Column Chromatography

The proteins of interest (such as Vp28 polypeptides) can also be separated from other proteins on the basis of their size, net surface charge, hydrophobicity and affinity for ligands. In addition, antibodies raised against Vp28 polypeptides can be conjugated to column matrices and the Vp28 polypeptides immunopurified. All of these methods are well known in the art.

It will be apparent to one of skill that chromatographic techniques can be performed at any scale and using equipment from many different manufacturers (e.g., Pharmacia Biotech).

B. Chemical Synthesis of Vp28 Polypeptides

Alternatively, Vp28 polypeptides of the present invention may be synthesized chemically using conventional peptide synthesis or other protocols well known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

III. Production of Antibodies to Vp28 polypeptides of the Invention

Antibodies against Vp28 polypeptides of the present invention can be obtained from a variety of sources. These antibodies may be naturally occurring antibodies that require isolation, purification, and preferably, quantification. These antibodies may also be artificial: they may be chimeric antibodies or antibodies recombinantly produced, including single chain antibodies (ScFv).

A. Naturally Occurring Antibodies

1. Production of Antibodies with Desired Specificity

Methods for producing polyclonal and monoclonal antibodies that react specifically with an immunogen of interest are known to those of skill in the art (see, e.g. Coligan, Current Protocols in Immunology Wiley/Greene, NY, 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256:495-497, 1975). Such techniques include antibody preparation by selection of antibodies from libraries of recombinant antibodies in phage or similar vectors (see, Huse et al., Science 246:1275-1281, 1989; and Ward et al., Nature 341:544-546, 1989).

In order to produce an antibody with desired specificity for a Vp28 polypeptide of this invention, a naturally occurring polypeptide, e.g., one comprising SEQ ID NO:3 or 4, may be isolated from WSSV infected cells and used to immunize suitable animals, e.g., mice, rabbits, or primates. A standard adjuvant, such as Freund's adjuvant, can be used in accordance with a standard immunization protocol. Alternatively, a synthetic peptide derived from that a Vp28 polypeptide can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. Further fractionation of the antisera to enrich antibodies specifically reactive to the antigen and purification of the antibodies can be accomplished subsequently, see, Harlow and Lane, supra, and general descriptions of antibody purification offered below.

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Furthermore, antibodies against Vp28 polypeptides of the present invention may be produced by eggs discharged from animals that have been immunized by administration of a Vp28 polypeptide. The preferred animals include birds, such as chickens (particularly laying hens), ducks, turkeys, etc. The Vp28 polypeptide may be delivered into animals by, e.g., intramuscular injection, subcutaneous injection, intravenous injection, or oral administration. The amount of polypeptide injected may vary from 10 μg to 1 mg or according to the conditions of the animal, and the polypeptide is administered repeatedly until the amount of antibody in yolk reaches its maximum. The antibodies against Vp28 polypeptide can be purified from the eggs according to conventional antibody isolation methods. The eggs themselves may be used as sources of antibodies in dried, powdered, or aqueous form. The detailed description may be found in WO 03/070258, which is incorporated hereby in the entirety.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. A more detailed description of antibody production by recombinant methods can be found in a later section.

2. Purification of Antibodies

Standard methods for protein purification, such as those described in an earlier section, are suitable for purification of antibodies against Vp28 polypeptides of the invention.

B. Artificially Produced Antibodies

1. General Approaches

Besides naturally-occurring antibodies, artificially produced antibodies may also be used to practice the present invention. The general methods for recombinantly producing antibodies with desired specificity are known to those skilled in the relevant art and are described in numerous publications. See, e.g., U.S. Pat. No. 5,665,570. Briefly, the genes encoding an antibody with desired specificity can be identified by screening a B cell cDNA library using various cloning techniques, e.g., a cloning method based on polymerase chain reaction (PCR), and subsequently expressed in suitable host cells. For a general description of recombinant DNA technology, see, e.g., Sambrook and Russell, Molecular Cloning: A Laboratory Manual 3d ed. 2001; Kriegler, Gene Transfer and Expression: A Laboratory Manual 1990; and Ausubel et al., Current Protocols in Molecular Biology 1994.

Another means for recombinantly producing antibodies with desired specificity relies on the chimeric antibody technology. Generally, the genes encoding the variable regions of a non-human monoclonal antibody (e.g., a murine antibody) are cloned and joined with the coding sequences for human constant regions to produce the so-called “humanized” antibodies. See, e.g., U.S. Pat. Nos. 5,502,167; 5,607,847; 5,773,247. Such humanized chimeric antibodies produced by host cells are suitable for constructing the claimed liquid IgG and IgM calibrators.

2. Transfection and Expression

Various transfection methods, host cell lines, and expression vectors are suitable for the expression of a recombinant antibody. Detailed description for these subjects can be found in an earlier section where recombinant production of Vp28 polypeptide is discussed.

3. Purification of Recombinant Antibodies

The recombinant antibodies may be purified to substantial purity by standard techniques as described above, including selective precipitation with such substances as ammonium sulfate; column chromatography, gel filtration, immunopurification methods, and others (see, e.g., U.S. Pat. No. 4,673,641; Scopes, Protein Purification: Principles and Practice, 1982; Sambrook and Russell, supra; and Ausubel et al., supra).

IV. Administration of Vp28 Polypeptides or Their Antibodies

A. Administration by Feeding

Vp28 polypeptides of the invention or their antibodies can be administered to the animals, e.g., shrimp, by feeding. In such embodiments, the polypeptide or antibody is preferably formulated in a manner that protects the polypeptide or antibody from degradation. A number of such formulations are described in the art. For example, a Vp28 polypeptide or a Vp28 antibody can be fed to the animals as a preparation in which the polypeptide or antibody is prepared as an emulsion, e.g., associated with oil-bodies. Such preparations have been described, e.g., in U.S. Pat. Nos. 5,948,682; 6,146,645; and 6,210,742.

The amount of Vp28 polypeptides or their antibodies administered by feeding can vary, but is typically present in an amount from about 0.5 grams to 500 grams/ton, and is often present in an amount from about 1 gram to 100 grams/ton, typically from 5 to 25 or 50 grams/ton of feed.

Vp28 polypeptides or their antibodies can be administered by feeding at any stage of growth. Preferably, the polypeptides or antibodies can be administered at any time after the animals leave the hatchery where they are likely to be exposed to WSSV.

The feed containing Vp28 polypeptides or their antibodies is provided to the shrimp at regular intervals to maintain protection. For example, for shrimp that are at stage PL15 or above, the feed is preferably given three to four times daily; for larger animals, the feed is given at least as often as twice daily. Typically, the feeding frequency is not less than once daily.

B. Administration by Recombinant Algae

An alternative method for administering Vp28 polypeptides or recombinant antibodies of the present invention is using a delivery system of recombinant algae, as described by U.S. Patent Application No. 20030022359, hereby incorporated in the entirety. Briefly, the delivery system is a transgenic algae that comprises a transgene which comprises a polynucleotide encoding at least one peptide, for example a Vp28 polypeptide, and a promoter for driving expression of the polynucleotide in the algae. Preferably, the transgene further comprises a terminator that terminates transcription, and all other genetic elements required for transcription. The transgenic algae preferably further expresses the peptide.

The delivery of the recombinant Vp28 polypeptide of antibody may be achieved by oral administration of a transgenic algae described above, or immersion of the animals being treated into a suspension comprising water and the transgenic algae.

### EXAMPLES

The following examples are provided by way of illustration only and not by way of limitation. Those of skill in the art will readily recognize a variety of non-critical parameters that could be changed or modified to yield essentially similar results.

### Example 1

### Expression of Viral Proteins and Protein Fragments

The four major nucleocapsid and envelope proteins from WSSV were evaluated. Each protein was modeled using the MacVector software package for primary and secondary structural motifs. Predictions based on the amino acid sequence of each protein were examined for secondary structural features using multiple predictive algorithms. The results from each of the predictive techniques were averaged and this information, along with additional predictive information on hydrophilicity, surface probability, flexibility, and antigenic index were used to select portions of each protein to be expressed in the fusion system. The portion of each viral protein that may potentially interact with a cellular receptor in the viral host is likely to be exposed on the surface of the protein. In addition, the interactive portion of each protein is likely to be contained on a single structural domain. By using the predictive information, likely portions of each viral protein that would be expected to interact with a cellular receptor were selected.

Proteins were expressed using the PurePro Caulobacter Expression Systems from Invitrogen Corporation. This systems has the potential for a very high level of production, approaching one gram of expressed protein per liter of culture media. This is an advantage, as large amounts of protein are required for commercial use. The system also secretes the protein into the culture media, where it can be readily concentrated and purified. Further, Caulobacter grows well in very inexpensive medium, thus reducing production costs.

The expression protocol was modified to employ standard fermentation equipment to make the expressed protein fusion as a secreted soluble protein, which eliminates or simplifies solubilization, renaturation, and purification of the expressed fusion proteins.

### Example 2

### Inhibition of WSSV Infection Using Vp28 Protein Fragments

Inhibition of WSSV infection using Vp28 fragments was performed as follows. A total of twelve 9-liter plastic aquaria (31 ppt salinity, 30° C.) are used to house the animals from the time they are received until the time the experiment is terminated. The tanks are distributed randomly between two separate rack systems, each with its own common water recirculation system. In addition to the test groups, two sentinel tanks and two positive control tanks are used to monitor the potential escape of the pathogen from the exposed tanks and to confirm the virulence of the virus, respectively.

Six experimental feeds were produced for use in the bioassay. Two viral fusion proteins, one containing a fragment of Vp28 and one containing a fragment of Vp35 were used alone or in combination at two different concentrations to prepare an extruded feed. Juvenile Penaeus vannamei were fed the experimental feed for 72 hours prior to infection of tissue.

WSSV infectivitiy is tested as follows. The water recirculation system is turned off and an amount of freshly prepared WSSV-positive shrimp tissue equal to 5% of the total biomass of the tank is added. The shrimp are allowed to feed on the infected tissue for 2 hours prior to the water recirculation system being restarted. Nearly all of the tissue is typically consumed within the first few minutes; however, the shrimp are incubated further in the still water for maximum contact. This process is performed on three consecutive days.

Following exposure of the shrimp to WSSV-infected tissue, water is exchanged at a rate of 4.5 liters per hours (1,200% change per day). Temperature is maintained at 30° C. Shrimp are continually fed either the experimental or control diet as appropriate following pathogen exposure. The animals are monitored twice daily for a period of 14 days for feeding pattern changes, altered behavior, morphological changes, and deaths. Moribund shrimp are removed from the tanks and frozen at −80° C. for subsequent PCR analysis. Upon termination on day 30, all surviving shrimp are counted, sacrificed and archived for subsequent PCR analysis.

The results demonstrated that shrimp fed a diet containing the expressed Vp28 fragment fusion protein protected shrimp from WSSV infection. An average of 80% of the shrimp in the tanks that received either 25 grams per ton or 5 grams per ton of the Vp28 fusion survived whereas less than 25% of the control shrimp survived. The Vp35 fusion proteins did not exhibit any protective effect against WSSV challenge. Those animals that received a mixture of the Vp28 and Vp35 fusion proteins in the feed also exhibited enhanced survival relative to controls.

### Example 3

### White Spot Syndrome Virus Challenge

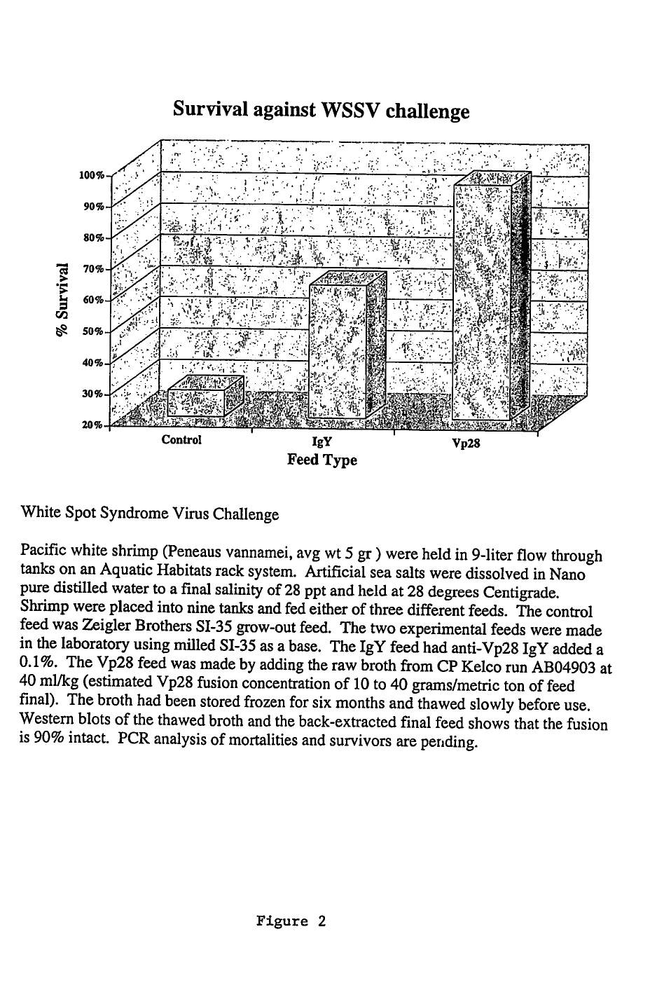

Pacific white shrimp (Peneaus vannamei, average weight 5 grams) were divided into groups and held in 9-liter flow through tanks on an Aquatic Habitats rack system. There were between 4 to 8 animals each tank, and 3 tanks in each group. Artificial sea salts were dissolved in Nano pure distilled water to a final salinity of 28 ppt and held at 28° C. Shrimp were placed in nine tanks and fed with one of three different feeds. The control feed was Zeigler Brothers SI-35 grow-out feed. The two experimental feeds were made in the laboratory using milled SI-35 as a base. The IgY feed had anti-Vp28 IgY added at 0.1%. The Vp28 feed was made by adding the raw both from CP Kelco run AB04903 at 40 ml/kg (estimated Vp28 fusion concentration of 10 to 40 grams/metric ton of feed final). In this experiment, the Vp28 fusion is a recombinant polypeptide of Vp28 fragment 1E (SEQ ID NO:4) fused with the surface array protein RsaA from Caulobacter cresentus produced by Invitrogen's PurePro Caulobacter Expression System. Anti-Vp28 IgY is an antibody against Vp28 fusion raised in chicken. The broth had been stored frozen for six months and thawed slowly before use. Western blots of the thawed broth and the back-extracted final feed shows that the fusion is 90% intact. The shrimp were challenged by exposure to WSSV as described in Example 2. The survival of different groups that had been given different feeds, 10 days after the initial WSSV exposure and 7 days after the final exposure, is shown in FIG. 2.

All patents, patent applications, and other publications cited in this application, including published amino acid or polynucleotide sequences, are incorporated by reference in the entirety for all purposes.

```
SEQ ID NO:1 nucleic acid encoding Vp28 CDS 323.937
Accession number AF173993
      1 aatgcaacca cccaagagag caaaacttct tccccaacaa tctcctcgac cccaactaca
    61 tattctggca gctcaaccag caggggtcca ggttctggat ctggaaacaa acccaaagat
  121 gacacatccg ttgaaggaat agaccctggc ttactgtaac agaaaaaaga gtaaaaggcg
  181 acagctcgct tgccaattgt cctgttacgt actctgtggt ttcacgaggt tgtcatcacc
  241 aaaggtaacc tttttttttg tcctcgccga caaaacgaca tcttaataac caagcaacgt
  301 tcgataaaga aaaaaactcg tcatggatct ttctttcact ctttcggtcg tgtcggccat
  361 cctcgccatc actgctgtga ttgctgtatt tattgtgatt tttaggtatc acaacactgt
  421 gaccaagacc atcgaaaccc acacagacaa tatcgagaca aacatggatg aaaacctccg
  481 cattcctgtg actgctgagg ttggatcagg ctacttcaag atgactgatg tgtcctttga
  541 cagcgacacc ttgggcaaaa tcaagatccg caatggaaag tctgatgcac agatgaagga
  601 agaagatgcg gatcttgtca tcactcccgt ggagggccga gcactcgaag tgactgtggg
  661 gcagaatctc acctttgagg gaacattcaa ggtgtggaac aacacatcaa gaaagatcaa
  721 catcactggt atgcagatgg tgccaaagat taacccatca aaggcctttg tcggtagctc
  781 caacacctcc tccttcaccc ccgtctctat tgatgaggat gaagttggca cctttgtgtg
  841 tggtaccacc tttggcgcac caattgcagc taccgccggt ggaaatcttt tcgacatgta
  901 cgtgcacgtc acctactctg gcactgagac cgagtaaata aatcgtgctt ttttatatag
  961 atagggaatt ttaatattac aacaataaga aaataaaaca attgaggaaa tttataccat
1021 attttattga cctacttaac cttcttgcta tacaatgaat gtttaagtga ctggaaaagt
1081 ttagcaatat tatccttgaa cgggaaacat gcaccaatta
```

SEQ ID NO:2 Vp28 full-length polypeptide sequence
MDLSFTLSVVSAILAITAVIAVFIVIFRYHNTVTKTIETHTDNIETNMDE
NLRIPVTAEVGSGYFKMTDVSFDSDTLGKIRNGKSDAQMEEDADLVITPV
EGRALEVTVGQNLTFEGTFKVWNNTSRKINITGMQMVPKINPSKAFVGSS
NTSSFTPVSIDEDEVGTFVCGTTFGAPIAATAGGNLFDMYVHVTYSGTET
E

SEQ ID NO:3 Vp28 polypeptide fragment 4C
(107-150 of SEQ ID NO:2, 44 a. a.)
ALEVTVGQNLTFEGTFKVWNNTSRKINITGMQMVPKINPSKAFV

SEQ ID NO:4 Vp28 polypeptide fragment 1E
(28-114 of SEQ ID NO:2, 87 a. a.)
RYHNTVTKTIETHTDNIETNMDENLRIPVTAEVGSGYFKMTDVSFDSDTL
GKIKIRNGKSDAQMKEEDADLVITPVEGRALEVTVGQ

SEQ ID NO:5 Vp28 polypeptide fragment 5A
(102-204 of SEQ ID NO:2, 103 a. a.)
PVEGRALEVTVGQNLTFEGTFKVWNNTSRKINITGMQMVPKINPSKAFVG
SSNTSSFTPVSIDEDEVGTFVCGTTFGAPIAATAGGNLFDMYVHVTYSGT
ETE

SEQ ID NO:6 Vp28 polypeptide fragment 6A
(150-204 of SEQ ID NO:2, 55 a. a.)
VGSSNTSSFTPVSIDEDEVGTFVCGTTFGAPIAATAGGNLFDMYVHVTYS
GTETE

SEQ ID NO:7 Vp28 polypeptide fragment 3E
(28-204 of SEQ ID NO:2, 177 a. a.)
RYHNTVTKTIETHTDNIETNMDENLRIPVTAEVGSGYFKMTDVSFDSDTL
GKIKIRNGKSDAQMKEEDADLVITPVEGRALEVTVGQNLTFEGTFEGTGK
VWNNTSRKINITGMQMVPKINPSKAFVGSSNTSSFTPVSIDEDEVGTFVC
GTTFGAPIAATAGGNLFDMYVHVTYSGTETE

SEQ ID NO:8 Vp28 fragment 2D
(28-150 of SEQ ID NO:2, 123 a. a.)
RYHNTVTKTIETHTDNIETNMDENLRIPVTAEVGSGYFKMTDVSFDSDTL
GKIKIRNGKSDAQMKEEDADLVITPVEGRALEVTVGQNLTFEGTFKVWNN
TSRKINITGMQMVPKINPSKAFV

What is claimed is:

1. An isolated polypeptide comprising a Vp28 peptide that consists of an amino acid sequence of at least 44 contiguous amino acids of positions 28-204 of SEQ ID NO:2, wherein the polypeptide inhibits White Spot Syndrome Virus (WSSV) infection of a crustacean.

2. The isolated polypeptide of claim 1, wherein the Vp28 peptide comprises at least 50 contiguous amino acids of positions 28-204 of SEQ ID NO:2.

3. The isolated polypeptide of claim 1, wherein the Vp28 peptide comprises at least 100 contiguous amino acids of positions 28-204 of SEQ ID NO:2.

4. The isolated polypeptide of claim 1, wherein the Vp28 peptide comprises SEQ ID NO:3, SEQ ID NO:4, SEQ ID NO:5, SEQ ID NO:6, or SEQ ID NO:8.

5. The isolated polypeptide of claim 1, wherein the Vp28 peptide consists of the amino acid sequence of SEQ ID NO:7.

6. The isolated polypeptide of claim 1, wherein the crustacean is a member of the genus Penaeus.

7. An isolated nucleic acid encoding a polypeptide comprising a Vp28 peptide that consists of at least 44 contiguous amino acids of positions 28-204 of SEQ ID NO:2, wherein the polypeptide inhibits White Spot Syndrome Virus (WSSV) infection of a crustacean.

8. The isolated nucleic acid of claim 7, wherein the Vp28 peptide comprises at least 50 contiguous amino acids of positions 28-204 of SEQ ID NO:2.

9. The isolated nucleic acid of claim 7, wherein the Vp28 peptide comprises at least 100 contiguous amino acids of positions 28-204 of SEQ ID NO:2.

10. The isolated nucleic acid of claim 7, wherein the Vp28 peptide comprises SEQ ID NO:3, SEQ ID NO:4, SEQ ID NO:5, SEQ ID NO:6, or SEQ ID NO:8.

11. The isolated nucleic acid of claim 7, wherein the Vp28 peptide consists of the amino acid sequence of SEQ ID NO:7.

12. The isolated nucleic acid of claim 7, wherein the crustacean is a member of the genus Penaeus.

13. A method for inhibiting White Spot Syndrome Virus (WSSV) infection of a crustacean, the method comprising administering to the crustacean a polypeptide comprising a Vp28 peptide that consists of an amino acid sequence of at least 44 contiguous amino acids of positions 28-204 of SEQ ID NO:2.

14. The method of claim 13, wherein the Vp28 peptide comprises at least 50 contiguous amino acids of positions 28-204 of SEQ ID NO:2.

15. The method of claim 13, wherein the Vp28 peptide comprises at least 100 contiguous amino acids of positions 28-204 of SEQ ID NO:2.

16. The method of claim 13, wherein the Vp28 peptide comprises SEQ ID NO:3, SEQ ID NO:4, SEQ ID NO:5, SEQ ID NO:6, or SEQ ID NO:8.

17. The method of claim 13, wherein the Vp28 peptide consists of the amino acid sequence of SEQ ID NO:7.

18. The method of claim 13, wherein the administering comprises feeding the polypeptide to the crustacean.

19. The method of claim 13, wherein the crustacean is a member of the genus Penaeus.

20. A method for inhibiting White Spot Syndrome Virus (WSSV) infection of a crustacean, the method comprising administering to the crustacean a polypeptide comprising the amino acid sequence of SEQ ID NO:2

21. A feed for a crustacean, wherein the feed comprises a polypeptide as set forth in claim 1 or a polypeptide comprising the amino acid sequence of SEQ ID NO:2.

22. The feed of claim 21, wherein the crustacean is a member of the genus Penaeus.
